(19) United States
(12) Patent Application Publication
(10) Pub. No.: US 2018/0369514 A1
(43) Pub. Date: Dec. 27, 2018

(54) Metered Dose Inhaler

(71) Applicant: Resolve Digital Health Inc., Toronto (CA)

(72) Inventor: Robert ADELSON, Vaughn (CA)

(73) Assignee: Resolve Digital Health Inc., Toronto (CA)

(21) Appl. No.: 16/020,497

(22) Filed: Jun. 27, 2018

Related U.S. Application Data
(60) Provisional application No. 62/525,543, filed on Jun. 27, 2017.

(51) Int. Cl. A61M 15/00

(52) U.S. Cl. CPC A61M 15/008; G16H 20/10; A61M 2205/52; A61M 15/009

(57) ABSTRACT

The present disclosure relates to an inhaler for dispensing metered doses of aerosolized medication from an aerosol container that contains a formulation for inhalation by a user, wherein the inhaler comprises one or more processors that are operatively coupled to a memory for monitoring and recording medication dose parameters based on consumption of the medication by the user. Exemplary dose parameters can include any or a combination of timing of dose consumption, size of each dose, frequency of doses, and effectiveness grade of the prescribed dose of medication, wherein the effectiveness grade is determined based on a feedback signal received from the user after the metered dose is consumed, wherein the feedback signal is received by the inhaler from any or a combination interface of the inhaler or from a portable computing device that the inhaler is operatively coupled with.

### TECHNICAL FIELD

The present disclosure relates to technical field of therapeutic inhalers. In particular, the present disclosure relates to a metered dose inhaler that is communicable and includes a means for monitoring medication dose and effectiveness of prescribed dose of medication.

### BACKGROUND

Background description includes information that may be useful in understanding the present invention. It is not an admission that any of the information provided herein is prior art or relevant to the presently claimed invention, or that any publication specifically or implicitly referenced is prior art.

Inhalation devices are generally used for delivering medication into body via lungs, and are widely used in the management of asthma or other respiratory diseases. Inhalation devices commonly comprise pressurized canister assembly in which medication particles are stored under pressure in a suspension or solution form. On activation, inhalation devices release a fixed or predetermined dose of medication in aerosol form through a mouthpiece for inhalation by a patient.

Various types of inhalation devices have been developed and used by patients with asthma or other respiratory disorders for delivering a controlled dosage of inhalation medications. However, the existing devices fail entirely to provide provisions for gathering information related to medication dose such as dose size, timing and frequency of doses, and for analysing the gathered data in a way that would be useful for either therapy or in a clinical setting. Furthermore, there is no mechanism to monitor the symptoms before and after a dose of inhalation medication has been taken i.e., effectiveness of a prescribed dose in a patient, and communicate the same to doctors, pharmaceutical companies, drug stores or caregiver in real time to enhance patient clinical care. Therefore, pharmaceutical companies, doctors or caregivers have no means to remain connected with the user to learn about the effectiveness of the medication.

Accordingly, it would be advantageous to provide an inhalation device with features to monitor administration of medication and medical treatment regimens thereby enhancing patient clinical care. Yet another advantage would be to provide an inhalation device that is communicable in a web enabled environment to implement an effective medication management system. Further, incorporation of smart features that can enable keeping of record of symptoms before and after a dose can help in allowing doctors and patients to gain a better understanding of how the medication is functioning to develop a customized treatment program.

The present invention satisfies the existing needs, as well as others, and generally overcomes the deficiencies found in the prior art.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all groups used in the appended claims.

### SUMMARY OF THE INVENTION

In an aspect, the present disclosure relates to an inhaler for dispensing metered doses of aerosolized medication from an aerosol container, said container containing a formulation for inhalation by a user, said inhaler comprising one or more processors operatively coupled to a memory so as to monitor and record medication dose parameters based on consumption of said medication by said user, wherein said dose parameters comprise any or a combination of timing of dose consumption, size of each dose, and frequency of doses in a defined duration, and effectiveness grade of the prescribed dose of medication, wherein said the effectiveness grade is determined based on a feedback signal received from the user after the metered dose is consumed, wherein the feedback signal is received by the inhaler from any or a combination interface of the inhaler or from a portable computing device that said inhaler is operatively coupled with, and wherein the feedback signal is indicative of how the user is feeling post consumption of the prescribed dose of medication.

In an aspect, the inhaler can dispense only a pre-defined amount of dose per iteration, wherein the pre-defined amount is configured through the portable computing device.

In another aspect, the portable computing device can be any or a combination of a smart phone, a mobile phone, a tablet PC, a wearable device, and a laptop PC.

In another aspect, the recorded medication dose parameters can, in real-time or at pre-defined time intervals, be encrypted and then transmitted from the inhaler to the portable computing device, wherein said portable computing device decrypts the recorded medication dose parameters for analysis of any or a combination user treatment monitoring, consumption pattern, symptoms before and after a prescribed dose, and user feedback based on which said portable computing device revises the treatment regime.

In an aspect, the inhaler can further include an aerosol medication dispensing unit for dispensing said metered doses of aerosolized medication for inhalation, and a housing having a receiving end configured to receive in the aerosol container, and a mouthpiece end, wherein the dispensing unit is configured such that the container is removably inserted into the housing, and each time the container is depressed, a constant predetermined dose of medication is discharged from the container through the mouthpiece in aerosol form for inhalation by the user.

In another aspect, the interface of the proposed inhaler can be a touch-panel based interface that allows said user to select any or a combination of dose type, dose size, reminder for dose consumption, dose formulation, and wherein said touch-panel based interface allows said user to wirelessly communicate with one or more portable computing devices and enable issuance of feedback signals, and receipt of customized treatment regimens for compliance during metered dose medication.

Aspects of the present disclosure relate to a smart inhaler capable of dispensing metered doses of aerosolized medication from an aerosol container containing pharmaceutical or herbal formulations for inhalation by a patient. The disclosed smart inhaler can monitor and record medication dose including timing, dose size and frequency of doses, and effectiveness of prescribed dose of medication.

In an aspect, the inhaler disclosed herein can include an aerosol medication dispensing module for dispensing metered doses of aerosolized medication for inhalation. The medication dispensing module can include a main body or housing including a receiving end configured to receive in an aerosol container, and a mouthpiece end. The medication dispensing module can be configured such that an aerosol container is removably inserted into the housing, and each time the container is depressed a constant predetermined dose of medication is discharged from the container through the mouthpiece in aerosol form for inhalation.

In an aspect, the inhaler disclosed herein can incorporate smart features to enable record keeping in respect of timing, dose size, frequency of doses and symptoms before and after a prescribed dose. The maintained records can help in allowing doctors and patients to gain a better understanding of how the medication is functioning to develop a customized treatment program. In an embodiment, the inhaler can include a display screen configured to display patient's symptoms before and after a prescribed dose has been taken.

In an aspect, the disclosed inhaler can further incorporate means to communicate data in real time or on as required basis to various stakeholders such as doctor, care giver, drug stores, pharmaceutical companies etc. The means for communication can be a wireless communication means that can enable data transfer from the inhaler to external functional units or networks. Communication between the inhaler and external functional units or networks may be accomplished through any suitable communication channels such as Bluetooth, Wi-Fi, GSM communication means such as 3G/4G/5G etc.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure.

FIG. 1 illustrates an exemplary block diagram indicating various functional parts/modules of a smart inhaler in accordance with embodiments of the present disclosure.

FIG. 2 illustrates an exemplary image of the inhaler in accordance with embodiments of the present disclosure.

### DETAILED DESCRIPTION OF THE INVENTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Unless the context requires otherwise, throughout the specification which follow, the word “comprise” and variations thereof, such as, “comprises” and “comprising” are to be construed in an open, inclusive sense that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

The headings and abstract of the invention provided herein are for convenience only and do not interpret the scope or meaning of the embodiments.

Various terms as used herein. To the extent a term used in a claim is not defined below, it should be given the broadest definition persons in the pertinent art have given that term as reflected in printed publications and issued patents at the time of filing.

In an aspect, the present disclosure relates to an inhaler for dispensing metered doses of aerosolized medication from an aerosol container, said container containing a formulation for inhalation by a user, said inhaler comprising one or more processors operatively coupled to a memory so as to monitor and record medication dose parameters based on consumption of said medication by said user, wherein said dose parameters comprise any or a combination of timing of dose consumption, size of each dose, and frequency of doses in a defined duration, and effectiveness grade of the prescribed dose of medication, wherein said the effectiveness grade is determined based on a feedback signal received from the user after the metered dose is consumed, wherein the feedback signal is received by the inhaler from any or a combination interface of the inhaler or from a portable computing device that said inhaler is operatively coupled with, and wherein the feedback signal is indicative of how the user is feeling post consumption of the prescribed dose of medication.

In an aspect, the inhaler can dispense only a pre-defined amount of dose per iteration, wherein the pre-defined amount is configured through the portable computing device.

In another aspect, the portable computing device can be any or a combination of a smart phone, a mobile phone, a tablet PC, a wearable device, and a laptop PC.

In another aspect, the recorded medication dose parameters can, in real-time or at pre-defined time intervals, be encrypted and then transmitted from the inhaler to the portable computing device, wherein said portable computing device decrypts the recorded medication dose parameters for analysis of any or a combination user treatment monitoring, consumption pattern, symptoms before and after a prescribed dose, and user feedback based on which said portable computing device revises the treatment regime.

In an aspect, the inhaler can further include an aerosol medication dispensing unit for dispensing said metered doses of aerosolized medication for inhalation, and a housing having a receiving end configured to receive in the aerosol container, and a mouthpiece end, wherein the dispensing unit is configured such that the container is removably inserted into the housing, and each time the container is depressed, a constant predetermined dose of medication is discharged from the container through the mouthpiece in aerosol form for inhalation by the user.

In another aspect, the interface of the proposed inhaler can be a touch-panel based interface that allows said user to select any or a combination of dose type, dose size, reminder for dose consumption, dose formulation, and wherein said touch-panel based interface allows said user to wirelessly communicate with one or more portable computing devices and enable issuance of feedback signals, and receipt of customized treatment regimens for compliance during metered dose medication.

Aspects of the present disclosure relate to an inhaler capable of dispensing metered doses of aerosolized medication from an aerosol container containing pharmaceutical or herbal formulations for inhalation by a patient. The disclosed smart inhaler can monitor and record medication dose including timing, dose size and frequency of doses, and effectiveness of prescribed dose of medication.

In an aspect, the inhaler disclosed herein can include an aerosol medication dispensing module for dispensing metered doses of aerosolized medication for inhalation. The medication dispensing module can include a main body or housing including a receiving end configured to receive in an aerosol container, and a mouthpiece end. The medication dispensing module can be configured such that an aerosol container is removably inserted into the housing, and each time the container is depressed a constant predetermined dose of medication is discharged from the container through the mouthpiece in aerosol form for inhalation.

In an aspect, the inhaler disclosed herein can incorporate smart features to enable record keeping in respect of timing, dose size, frequency of doses and symptoms before and after a prescribed dose. The maintained records can help in allowing doctors and patients to gain a better understanding of how the medication is functioning to develop a customized treatment program. In an embodiment, the inhaler can include a display screen configured to display patient's symptoms before and after a prescribed dose has been taken.

In an aspect, the disclosed inhaler can further incorporate means to communicate data in real time or on as required basis to various stakeholders such as doctor, care giver, drug stores, pharmaceutical companies etc. The means for communication can be a wireless communication means that can enable data transfer from the inhaler to external functional units or networks. Communication between the inhaler and external functional units or networks may be accomplished through any suitable communication channels such as Bluetooth, Wi-Fi, GSM communication means such as 3G/4G/5G etc.

According to one aspect of the present disclosure, there is provided an inhaler capable of dispensing metered amounts or metered doses of medication from an aerosol container for inhalation by a patient. The proposed inhaler disclosed herein can incorporate features that enable record keeping in respect of timing, dose size, frequency of doses, and symptoms before and after a prescribed dose. The maintained records can help in allowing doctors and patients to gain a better understanding of how the medication is functioning to develop a customized treatment program. The smart inhaler can further incorporate means to communicate data, in real time or on as required basis, to various stakeholders such as doctor, pharmaceutical companies, drug stores, caregiver etc.

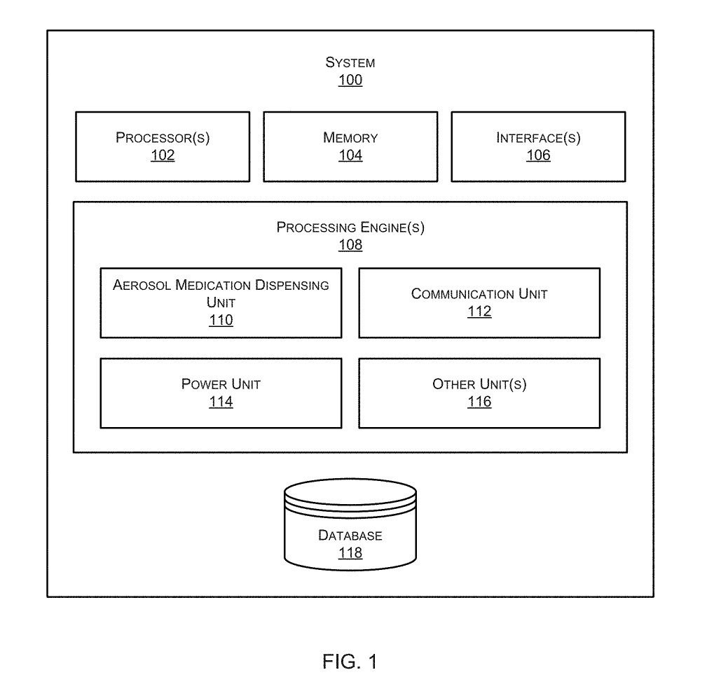

Referring now to FIG. 1, there is shown an exemplary block diagram 100 indicating various functional subsystems of the smart inhaler in accordance with embodiments of the present disclosure. As illustrated, the proposed inhaler device/system 100 can include one or more processor(s) 102 (also interchangeably referred to as micro-controllers). The one or more processor(s) 102 can be implemented as one or more microprocessors, microcomputers, microcontrollers, digital signal processors, central processing units, logic circuitries, and/or any devices that manipulate data based on operational instructions. Among other capabilities, the one or more processor(s) 102 are configured to fetch and execute computer-readable instructions stored in a memory 104 of the system 100. The processors 102 can further be configured to receive instructions from a portable computing device such as a smart phone that may be operatively coupled with the inhaler 100, and accordingly follow the dispensing process through vaporization of liquid(s) contained in the inhaler 100, wherein the vaporization, dispensing, drop generation, among other functional aspects of the proposed inhaler 100 can be configured/controller/monitored/analyzed at the portable computing device.

The memory 104 can store one or more computer-readable instructions or routines, which may be fetched and executed to create or share the data units over a network service. The memory 104 can include any non-transitory storage device including, for example, volatile memory such as RAM, or non-volatile memory such as EPROM, flash memory, and the like. In an example embodiment, the memory 104 may be a local memory or may be located remotely, such as a server, a file server, a data server, and the Cloud.

The inhaler 100 can also include an interface(s) 106. The interface(s) 106 may include a variety of interfaces, for example, interfaces for data input and output devices, referred to as I/O devices, storage devices, and the like. The interface(s) 106 may facilitate communication of the system 100 with various devices coupled to the system 100. The interface(s) 106 may also provide a communication pathway for one or more components of the system 100. Examples of such components include, but are not limited to, processing engine(s) 108 and data 118.

inhaler The engine(s) 108 can be implemented as a combination of hardware and programming (for example, programmable instructions) to implement one or more functionalities of the engine(s) 108. In examples described herein, such combinations of hardware and programming may be implemented in several different ways. For example, the programming for the engine(s) may be processor executable instructions stored on a non-transitory machine-readable storage medium and the hardware for the engine(s) 108 may include a processing resource (for example, one or more processors), to execute such instructions. In the present examples, the machine-readable storage medium may store instructions that, when executed by the processing resource, implement the engine(s) 108. In such examples, the system 100 can include the machine-readable storage medium storing the instructions and the processing resource to execute the instructions, or the machine-readable storage medium may be separate but accessible to system 100 and the processing resource. In other examples, the engine(s) 108 may be implemented by electronic circuitry. The data 118 can include data that is either stored or generated as a result of functionalities implemented by any of the components of the engine(s) 108.

The inhaler 100 can include an aerosol medication dispensing unit 110, a communication unit 112, a power unit 114, and one or more additional/other units 116.

According to embodiments of the present disclosure, the aerosol medication dispensing module/unit/section 110 can include a main body or housing including a receiving end configured to receive in an aerosol container, and a mouthpiece end. The housing can be tubular housing that can have a geometrically shaped cross-section such as, but not limited to circular, square, rectangular, oval, etc. An aerosol container, after insertion into the housing, can be depressed to discharge metered doses of aerosolized medication through the mouthpiece end for inhalation by a patient. The medication dispensing unit 110 can be configured such that an aerosol container is removably inserted into the housing, and each time the container is depressed a constant predetermined dose of medication is discharged from the container through the mouthpiece in aerosol form for inhalation. The aerosol medication dispensing unit 102 can further include a cap that can be configured to cover the mouthpiece to prevent contaminants from entering the interior of the dispensing module 110 and to keep the mouthpiece clean.

In an embodiment, the medication dispensing unit 110 can be configured to dispense a fixed dose of aerosolized medication or alternatively can be configured to vary the dose depending on symptoms or certain physiological parameters of the user.

In an embodiment, display interface 106 of the proposed inhaler can be configured to permit user to interact with the proposed inhaler to observe medication dose history including timing, dose size, and frequency of doses, dose effectiveness, account settings, and the like, and may also permit the user to control dose size, issue commands, to connect the inhaler with external functional units, and the like. In an embodiment, the interface 106 can be a touch screen that can be disposed on the housing of the aerosol medication dispensing module 102, and configured to display patient's symptoms before and after a prescribed dose has been taken. In another aspect, the interface can be a combination of buttons/actuators in conjunction with a touch panel or can function independently to enable one or more operational features including configuration of the proposed inhaler.

In an embodiment, database 118 can be configured to store information such as timing, dose size, frequency of doses, symptoms and/or physiological parameters before and after a prescribed dose etc. The stored information can provide records to help doctors and patients to gain a better understanding of how the medication is functioning to develop a customized treatment program. It can also allow pharmaceutical and nutraceutical companies to learn about the effectiveness of their medicine after clinical trials and can further allow them to stay connected to the patients using their medicines to gather data and perform analytics.

In an embodiment, the communication module 112 can be configured to communicate data in real time or on as required basis to various stakeholders such as doctor, caregiver, drug stores, pharmaceutical companies etc. The means for communication can be a wireless communication means that can enable data transfer from the inhaler 100 to external functional units or networks such as, but not limited to computing devices, mobile devices, networks, databases, other types of application software, etc. Communication between the inhaler 100 and external functional units or networks may be accomplished through any suitable communication channels such as, but not limited to, Bluetooth, Wi-Fi, GSM communication means such as 3G etc. Data transfer from the inhaler 100 to another functional unit can be in real time or as and when required.

In an embodiment, processer(s) 102 can be functionally coupled to the inhaler 100 and configured to control function of the inhaler 100, and the various modules and components thereof. In an exemplary embodiment, the processer(s) 102 can be configured to process digital data provided by user in respect of physiological parameters, symptoms etc. and based on pre-fed data provide dose to be administered.

In an embodiment, the power module 114 can be configured to meet power requirement of various functional subsystems of the smart inhaler. The power module 114 can include a rechargeable battery with connected charging port and charging circuit.

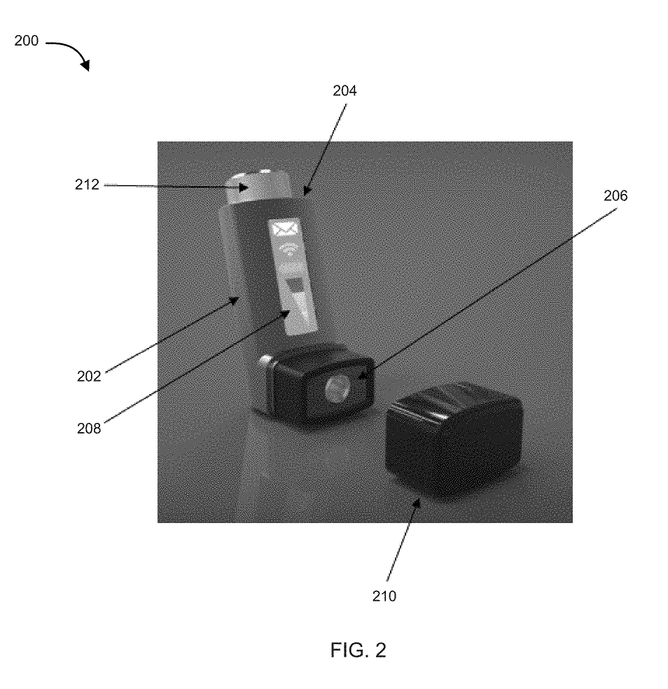

FIG. 2 illustrates an exemplary image 200 of the smart inhaler in accordance with embodiments of the present disclosure. As shown, the housing 202 of the inhaler can include a mouthpiece end 206 and a receiving end 204. The receiving end is adapted to receive a pressurized aerosol container 212 containing a predetermined number of unit doses of medication. The aerosol container 212 can be depressed to discharge metered doses of medication in aerosol form through the mouthpiece 206 for inhalation by a patient. The exemplary screen 208 provided on the housing 202 of the inhaler can indicate status of Wi-Fi, GSM connectivity, effectiveness of medication dose and battery status. The screen 208 can be further used to display and/or enter different data and information that the inhaler may provide or may be required to process or store. The cap 210 can be used to cover the mouthpiece 206 to prevent contaminants from entering the interior of the inhaler and to keep the mouthpiece clean.

In an aspect, the present disclosure relates to an inhaler for dispensing metered doses of aerosolized medication from an aerosol container, wherein the container can include a formulation (such as a medicated oil or a plant extract such as cannabis extract) for inhalation by a user. The inhaler can further include one or more processors that can be operatively coupled to a memory so as to monitor and record medication dose parameters based on consumption of the medication by the user, wherein the dose parameters can include any or a combination of timing of dose consumption, size of each dose, and frequency of doses in a defined duration, and effectiveness grade of the prescribed dose of medication, wherein said the effectiveness grade is determined based on a feedback signal received from the user after the metered dose is consumed, wherein the feedback signal can be received by the inhaler from any or a combination interface of the inhaler or from a portable computing device that said inhaler is operatively coupled with, and wherein the feedback signal is indicative of how the user is feeling post consumption of the prescribed dose of medication.

In an aspect, the user can, upon authentication through, for instance, a biometric, access the proposed inhaler device and then using the touch panel interface give his/her feedback on how he/she felt after the medication, said feedback being configured, for instance, in the form a set of multiple choice questions asked on the interface of the inhaler, such that based on receipt of the feedback inputs from the user, the inhaler can either process the feedback at its own end using execution of the instructions stored in the memory by the one or more processors and accordingly analyse how the treatment is impacting the user or how much dose (quantity, composition) is having what impact on the user. Any other parameter that the user may wish to give can also be configured as part of the instructions stored in the memory of the inhaler.

As the proposed inhaler is communicatively coupled with at least one portable computing device, the feedback can also be, in real-time, relayed back to the computing device and analysed there so as to generate strong analytics on how the user is reacting to the medication as well as enable an administrator to pull out analytics based on numerous such dose consumption records that a central computing device that he/she has access to receives from a plurality of inhalers.

Therefore, in an aspect, apart from allow a portable computing device to control and change dosing parameters of the instant inhaler, the proposed system can further include a central computing device such as a server/cloud that can receive multiple dose consumption records from multiple users having respective inhalers, based on which analytics can be performed on data sets of numerous inhalers, giving strong insights on treatment regimes, impact on user, behaviours change in user, among any other dosing parameter/attribute that may need to be monitored.

In an aspect, the inhaler can dispense only a pre-defined amount of dose per iteration, wherein the pre-defined amount is configured through the portable computing device.

In another aspect, the recorded medication dose parameters can, in real-time or at pre-defined time intervals, be encrypted and then transmitted from the inhaler to the portable computing device, wherein said portable computing device decrypts the recorded medication dose parameters for analysis of any or a combination user treatment monitoring, consumption pattern, symptoms before and after a prescribed dose, and user feedback based on which said portable computing device revises the treatment regime.

In an aspect, the inhaler can further include an aerosol medication dispensing unit for dispensing said metered doses of aerosolized medication for inhalation, and a housing having a receiving end configured to receive in the aerosol container, and a mouthpiece end, wherein the dispensing unit is configured such that the container is removably inserted into the housing, and each time the container is depressed, a constant predetermined dose of medication is discharged from the container through the mouthpiece in aerosol form for inhalation by the user.

In another aspect, the interface of the proposed inhaler can be a touch-panel based interface that allows said user to select any or a combination of dose type, dose size, reminder for dose consumption, dose formulation, and wherein said touch-panel based interface allows said user to wirelessly communicate with one or more portable computing devices and enable issuance of feedback signals, and receipt of customized treatment regimens for compliance during metered dose medication.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

We claim:

1. An inhaler for dispensing metered doses of aerosolized medication from an aerosol container, said container containing a formulation for inhalation by a user, said inhaler comprising one or more processors operatively coupled to a memory so as to monitor and record medication dose parameters based on consumption of said medication by said user, wherein said dose parameters comprise any or a combination of timing of dose consumption, size of each dose, and frequency of doses in a defined duration, and effectiveness grade of the prescribed dose of medication, wherein said the effectiveness grade is determined based on a feedback signal received from the user after the metered dose is consumed, wherein the feedback signal is received by the inhaler from any or a combination interface of the inhaler or from a portable computing device that said inhaler is operatively coupled with, and wherein the feedback signal is indicative of how the user is feeling post consumption of the prescribed dose of medication.

2. The inhaler of claim 1, wherein said inhaler dispenses only a pre-defined amount of dose per iteration, said pre-defined amount being configured through the portable computing device.

3. The inhaler of claim 1, wherein said portable computing device is any or a combination of a smart phone, a mobile phone, a tablet PC, a wearable device, and a laptop PC.

4. The inhaler of claim 1, wherein the recorded medication dose parameters are, in real-time or at pre-defined time intervals, encrypted and then transmitted from the inhaler to the portable computing device, wherein said portable computing device decrypts the recorded medication dose parameters for analysis of any or a combination user treatment monitoring, consumption pattern, symptoms before and after a prescribed dose, and user feedback based on which said portable computing device revises the treatment regime.

5. The inhaler of claim 1, wherein said inhaler further comprises:
an aerosol medication dispensing unit for dispensing said metered doses of aerosolized medication for inhalation.
a housing having a receiving end configured to receive in the aerosol container, and a mouthpiece end, wherein the dispensing unit is configured such that the container is removably inserted into the housing, and each time the container is depressed, a constant predetermined dose of medication is discharged from the container through the mouthpiece in aerosol form for inhalation by the user.

6. The inhaler of claim 1, wherein the interface of the inhaler is a touch-panel based interface that allows said user to select any or a combination of dose type, dose size, reminder for dose consumption, dose formulation, and wherein said touch-panel based interface allows said user to wirelessly communicate with one or more portable computing devices and enable issuance of feedback signals, and receipt of customized treatment regimens for compliance during metered dose medication.
